(19) United States
(12) Patent Application Publication
(10) Pub. No.: US 2024/0379189 A1
(43) Pub. Date: Nov. 14, 2024

(54) SYSTEMS AND METHODS FOR GENERATING NUCLEOTIDE SEQUENCE SYNTHESIS RELATED METRICS

(71) Applicant: Nutcracker Therapeutics, Inc., Emeryville, CA (US)

(72) Inventors: Robyn Beckwith, Berkeley, CA (US); Samuel Deutsch, Walnut Creek, CA (US); Evan McCartney-Melstad, Emeryville, CA (US); Sangeeta Nath, Fremont, CA (US)

(73) Assignee: Nutcracker Therapeutics, Inc., Emeryville, CA (US)

(21) Appl. No.: 18/692,189

(22) PCT Filed: Sep. 16, 2022
(86) PCT No.: PCT/US2022/043752

§ 371 Date: Mar. 14, 2024

Related U.S. Application Data
(60) Provisional application No. 63/245,528, filed on Sep. 17, 2021.

(51) Int. Cl. G16B 30/10

(52) U.S. Cl. CPC G16B 30/10

(57) ABSTRACT

Provided in one example is a system that includes one or more processors to receive a sequence data structure; apply at least one first metric of a plurality of metrics to the sequence data structure to generate at least one first metric score; determine that the at least one first metric score satisfies a first condition; apply, responsive to the at least one first metric score satisfying the first condition, at least one second metric of the plurality of metrics to the sequence data structure to generate at least one second metric score; and output an indication of the at least one first metric score and the at least one second metric score.

### CROSS-REFERENCE TO RELATED APPLICATIONS

The present application claims the benefit of and priority to U.S. Provisional Application No. 63/245,528, filed Sep. 17, 2021, the disclosure of which is incorporated herein by reference in its entirety.

### BACKGROUND

Nucleotide sequences, such as mRNA sequences, can be manufactured to have various properties. Errors can arise in the manufacturing process that can result in the manufactured nucleotide sequences not matching a target sequence.

### SUMMARY

Some examples provided herein relates generally to the field of nucleotide sequences. More particularly, the present disclosure relates to systems and methods for generating nucleotide sequence synthesis related metrics.

At least one aspect relates to a method of generating nucleic acid sequencing metrics. The method can include receiving, using one or more processors, a sequence data structure; applying, using the one or more processors, at least one first metric of a plurality of metrics to the sequence data structure to generate at least one first metric score; determining, using the one or more processors, that the at least one first metric score satisfies a first condition; applying, using the one or more processors, responsive to determining that the at least one first metric scores satisfies the first condition, at least one second metric of the plurality of metrics to the sequence data structure to generate at least one second metric score; and outputting, using the one or more processors, an indication of the at least one first metric score and the at least one second metric score.

The method can include detecting, by the one or more processors, at least one unique molecular index of the sequence data structure, and generating, by the one or more processors, the at least one second metric score using the at least one unique molecular index.

Generating the at least one second metric score can include adjusting the at least one second metric score to reduce an error rate associated with the sequence data structure.

The at least one unique molecular index can be adjacent to an i7 index of the sequence data structure.

The method can include identifying, by the one or more processors, at least one synthetic adapter sequence of the sequence data structure, removing, by the one or more processors, the at least one synthetic adapter sequence from the sequence data structure, merging, by the one or more processors responsive to removing the at least one synthetic adapter sequence, a plurality of unique molecular indices of the sequence data structure from which the at least one synthetic adapter sequence is removed, and generating, by the one or more processors, responsive to merging the plurality of unique molecular indices, the at least one second metric score to include a mismatch rate.

The subset of the plurality of metrics can include at least one metric of a flow cell used to generate the sequence data structure.

The method can include outputting, by the one or more processors, an indication of a failure condition responsive to the at least one first metric score not satisfying the first condition.

Outputting the indication can include generating, by the one or more processors, the indication to include instructions for modifying synthesis of a target sequence from which the sequence data structure is detected.

The method can include modifying synthesis of the target sequence using the instructions.

The at least one second metric score can include a target mismatch rate.

The sequence data structure can be from parallel sequencing of a nucleic acid.

The method can include generating an mRNA therapeutic using the indication.

Generating the mRNA therapeutic using the indication can include controlling, using the one or more processors, operation of a processor chip to reduce a difference between an mRNA of the mRNA therapeutic and a target sequence of the mRNA using the indication.

The method can include encapsulating at least one mRNA with at least one delivery vehicle composition to form the mRNA therapeutic.

The method can include generating a report containing the indication.

The method can include modifying a predetermined mRNA generation process using the indication.

At least one aspect relates to a system. The system can include one or more processors to receive a sequence data structure; apply at least one first metric of a plurality of metrics to the sequence data structure to generate at least one first metric score; determine that the at least one first metric score satisfies a first condition; apply, responsive to the at least one first metric score satisfying the first condition, at least one second metric of the plurality of metrics to the sequence data structure to generate at least one second metric score; and output an indication of the at least one first metric score and the at least one second metric score.

The one or more processors can be to detect at least one unique molecular index of the sequence data structure, and generate the at least one second metric score using the at least one unique molecular index.

The one or more processors can be to generate the at least one second metric score by adjusting the at least one second metric score to reduce an error rate associated with the sequence data structure.

The at least one molecular index can be adjacent to an i7 index of the sequence data structure.

The one or more processors can be to identify at least one synthetic adapter sequence of the sequence data structure, remove the at least one synthetic adapter sequence from the sequence data structure, merge, responsive to removing the at least one synthetic adapter sequence, a plurality of unique molecular indices of the sequence data structure from which the at least one synthetic adapter sequence is removed, and generate, responsive to merging the plurality of unique molecular indices, the at least one second metric score to include a mismatch rate.

The subset of metrics can include at least one metric of a flow cell used to generate the sequence data structure.

The one or more processors can be to output an indication of a failure condition responsive to the at least one first metric score not satisfying the first condition.

The one or more processors can be to generate the indication to include instructions for modifying synthesis of a target sequence from which the sequence data structure is detected.

The one or more processors can be to control operation of a processor chip to synthesize the target sequence using the instructions.

The at least one second metric score can include a target mismatch rate.

The sequence data structure can be from parallel sequencing of a nucleic acid.

The one or more processors can be to control operation of a processor chip to generate an mRNA therapeutic using the indication.

The one or more processors can be to execute a process to encapsulate at least one mRNA with at least one delivery vehicle composition to form the mRNA therapeutic.

The one or more processors can be to generate a report containing the indication.

The one or more processors can be to modify a predetermined mRNA generation process using the indication.

At least one aspect relates to a sequencing device. The sequencing device can include a sequencer to generate a sequence data structure based on a flow cell including a target sequence, and one or more processors to receive a sequence data structure; apply at least one first metric of a plurality of metrics to the sequence data structure to generate at least one first metric score; determine that the at least one first metric score satisfies a first condition; apply, responsive to the at least one first metric score satisfying the first condition, at least one second metric of the plurality of metrics to the sequence data structure to generate at least one second metric score; and output an indication of the at least one first metric score and the at least one second metric score.

The one or more processors can be to detect at least one unique molecular index of the sequence data structure, and generate the at least one second metric score using the at least one unique molecular index.

The one or more processors can be to generate the at least one second metric score by adjusting the at least one second metric score to reduce an error rate associated with the sequence data structure.

The at least one molecular index can be adjacent to an i7 index of the sequence data structure.

The one or more processors can be to identify at least one synthetic adapter sequence of the sequence data structure, remove the at least one synthetic adapter sequence from the sequence data structure, merge, responsive to removing the at least one synthetic adapter sequence, a plurality of unique molecular indices of the sequence data structure from which the at least one synthetic adapter sequence is removed, and generate, responsive to merging the plurality of unique molecular indices, the at least one second metric score to include a mismatch rate.

The subset of metrics can include at least one metric of a flow cell used to generate the sequence data structure.

The one or more processors can be to output an indication of a failure condition responsive to the at least one first metric score not satisfying the first condition.

The one or more processors can be to generate the indication to include instructions for modifying synthesis of a target sequence from which the sequence data structure is detected.

The one or more processors can be to control operation of a processor chip to synthesize the target sequence using the instructions.

The at least one second metric score can include a target mismatch rate.

The sequence data structure can be from parallel sequencing of a nucleic acid.

The one or more processors can be to control operation of a processor chip to generate an mRNA therapeutic using the indication.

The one or more processors can be to execute a process to encapsulate at least one mRNA with at least one delivery vehicle composition to form the mRNA therapeutic.

The one or more processors can be to generate a report containing the indication.

The one or more processors can be to modify a predetermined mRNA generation process using the indication.

At least one aspect relates to a non-transient processor-readable medium. The computer-readable medium can include computer-readable instructions that when executed by one or more processors cause the one or more processors to receive a sequence data structure; apply at least one first metric of a plurality of metrics to the sequence data structure to generate at least one first metric score; determine that the at least one first metric score satisfies a first condition; apply, responsive to the at least one first metric score satisfying the first condition, at least one second metric of the plurality of metrics to the sequence data structure to generate at least one second metric score; and output an indication of the at least one first metric score and the at least one second metric score.

The processor-readable medium can include instructions to cause the one or more processors to detect at least one unique molecular index of the sequence data structure and generate the at least one second metric score using the at least one unique molecular index.

The processor-readable medium can include instructions to cause the one or more processors to cause the one or more processors to generate the at least one second metric score by adjusting the at least one second metric score to reduce an error rate associated with the sequence data structure.

The at least one molecular index can be adjacent to an i7 index of the sequence data structure.

The processor-readable medium can include instructions to cause the one or more processors to identify at least one synthetic adapter sequence of the sequence data structure, remove the at least one synthetic adapter sequence from the sequence data structure, merge, responsive to removing the at least one synthetic adapter sequence, a plurality of unique molecular indices of the sequence data structure from which the at least one synthetic adapter sequence is removed, and generate, responsive to merging the plurality of unique molecular indices, the at least one second metric score to include a mismatch rate.

The processor-readable medium can include instructions to cause the one or more processors to detect at least one unique molecular index of the sequence data structure, and generate the at least one second metric score using the at least one unique molecular index.

The processor-readable medium can include instructions to cause the one or more processors to generate the at least one second metric score by adjusting the at least one second metric score to reduce an error rate associated with the sequence data structure.

The at least one molecular index can be adjacent to an i7 index of the sequence data structure.

The processor-readable medium can include instructions to cause the one or more processors to identify at least one synthetic adapter sequence of the sequence data structure, remove the at least one synthetic adapter sequence from the sequence data structure, merge, responsive to removing the at least one synthetic adapter sequence, a plurality of unique molecular indices of the sequence data structure from which the at least one synthetic adapter sequence is removed, and generate, responsive to merging the plurality of unique molecular indices, the at least one second metric score to include a mismatch rate.

The subset of metrics can include at least one metric of a flow cell used to generate the sequence data structure.

The processor-readable medium can include instructions to cause the one or more processors to output an indication of a failure condition responsive to the at least one first metric score not satisfying the first condition.

The processor-readable medium can include instructions to cause the one or more processors to generate the indication to indicate modifying synthesis of a target sequence from which the sequence data structure is detected.

The processor-readable medium can include instructions to cause the one or more processors to control operation of a processor chip for synthesizing of the target sequence using the indication.

The at least one second metric score can include a target mismatch rate.

The sequence data structure can be from parallel sequencing of a nucleic acid.

The processor-readable medium can include instructions to cause the one or more processors to control operation of a processor chip to generate an mRNA therapeutic using the indication.

The processor-readable medium can include instructions to cause the one or more processors to control operation of the processor chip to reduce a difference between an mRNA of the mRNA therapeutic and a target sequence of the mRNA using the indication.

The processor-readable medium can include instructions to cause the one or more processors to execute a process to encapsulate at least one mRNA with at least one delivery vehicle composition to form the mRNA therapeutic.

The processor-readable medium can include instructions to cause the one or more processors to generate a report containing the indication.

The processor-readable medium can include instructions to cause the one or more processors to modify a predetermined mRNA generation process using the indication.

At least one aspect relates to a system. The system can include a microfluidic path device to generate a nucleic acid; and a controller that includes one or more processors to receive an indication of an error of a target sequence associated with the nucleic acid relative to a reference sequence; and control operation of the microfluidic path device using the indication.

The controller can be to control operation of the processor chip to reduce a difference between the sequence data structure and the target sequence using the indication.

The controller can be to control operation of the processor chip using the indication by identifying an association between the error and a particular reagent of a plurality of reagents used to generate the nucleic acid and causing the processor chip to modify an amount of the particular reagent used to generate the nucleic acid.

The controller can be to control operation of the processor chip using the indication by determining, from the indication, that the error is associated with a particular contaminant, and causing the processor chip to provide the nucleic acid to a purification path of the processor chip targeted to remove the particular contaminant from the nucleic acid.

The nucleic acid can be a first nucleic acid, the processor chip can be to generate a product comprising the first nucleic acid and at least one second nucleic acid, and the controller can be to control operation of the processor chip using the indication by determining, from the indication, a difference between a ratio of the first nucleic acid to the at least one second nucleic acid and a target ratio of the first nucleic acid to the at least one second nucleic acid, and modifying operation of the processor chip to reduce the difference.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

It should be appreciated that all combinations of the foregoing concepts and additional concepts discussed in greater detail below are contemplated as being part of the inventive subject matter disclosed herein and may be employed in any combination to achieve the benefits described herein.

### BRIEF DESCRIPTION OF THE DRAWINGS

FIG. 1 is a block diagram of an example of a nucleic acid manufacturing system.

FIG. 2 is a block diagram of an example of a nucleic acid sequencer.

FIG. 3 is a block diagram of an example of a nucleic acid metric generator.

FIG. 4 is a flow diagram of an example of a method of generating nucleic acid sequencing metrics.

FIG. 5 is a block diagram of an example of a nucleic acid manufacturing system.

### DETAILED DESCRIPTION

Systems, apparatuses, and methods as described herein can be used to accurately detect errors in sequencing data of nucleic acids, including errors resulting from analytics processes, polymerase chain reaction (PCR), sequencing, or underlying biological differences between the nucleic acids (including mRNA) and target sequences that the nucleic acids are intended to be synthesized as. For example, various metrics can be determined from the sequencing data in order to identify and remove the errors. The metrics can be determined in a particular order to make the metric determination process more efficient, such as to more rapidly identify sequencing errors and trigger actions to address errors, including by reducing computations performed for evaluating failed samples or sequencing runs. Actions can be triggered in response to the metrics (and the underlying errors detected using the metrics), such as modifying how the nucleic acids are synthesized using the metrics. Particular metrics can be determined based on unique molecular indices (UMIs) included in the nucleic acids, which can facilitate distinguishing analytic differences between the nucleic acids and target sequences from biological differences between the nucleic acids and biological differences. For example, mRNA manufacturing quality control can be performed and improved by using the UMIs to identify sequence data corresponding to nucleic acids having the same UMI, and determining an error detected from the sequence data structure to correspond to an analytical error rather than an underlying biological error.

### A. Nucleotide Sequence Manufacturing

Nucleotide sequences, such as nucleic acids including mRNA, can be manufactured or synthesized for use in various applications, including for therapeutic delivery to a subject. For example, therapies such as mRNA therapeutics can be used for multiple treatment modalities including vaccination, immunotherapies, protein replacement therapies, tissue re-modelling/regeneration and treatment of genetic disease by gene editing. The mRNA can be manufactured to have a target sequence corresponding to a target protein, such as a target protein to perform a particular therapy. A double stranded DNA sequence can be used as a template for the transcription of the mRNA (e.g., by in vitro transcription (IVT)).

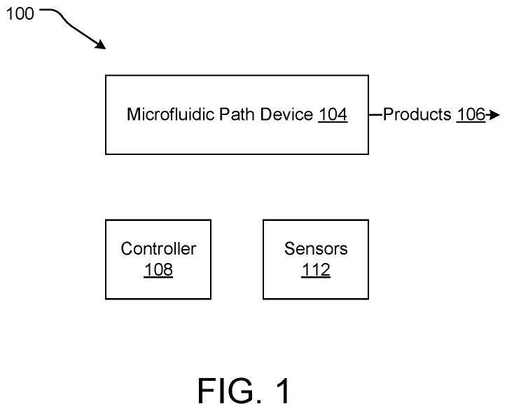

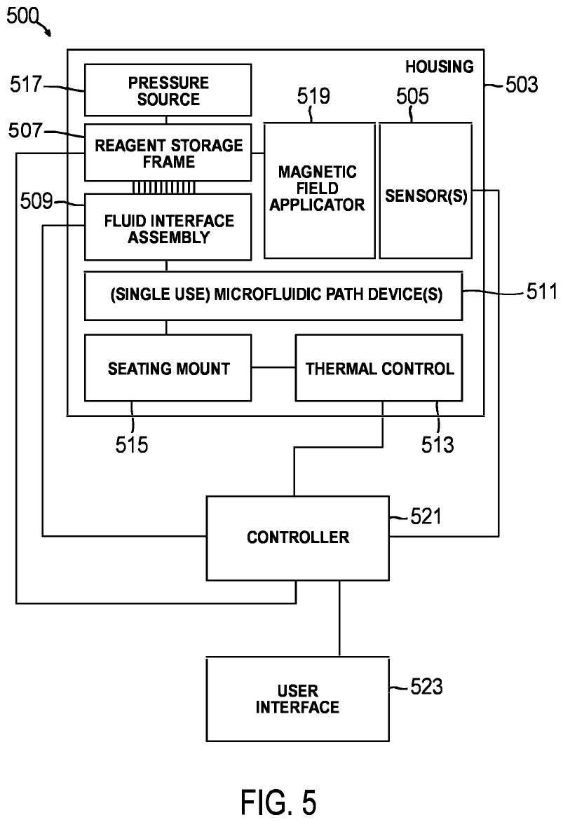

FIG. 1 depicts an example of a nucleic acid manufacturing system 100. The nucleic acid manufacturing system 100 can be used to manufacture a target mRNA sequence, including for therapeutic delivery to a subject. The nucleic manufacturing system 100 can be implemented using one or more components of the system 500 described with reference to FIG. 5.

The nucleic acid manufacturing system 100 can include at least one processor chip 104, such as a microfluidic path device or biochip. The processor chip 104 can include a plurality of reactors that receive reagents (e.g., via fluid channels) and cause reactions to be performed using the received reagents to generate target products 106. For example, various processor chips 104 can operate as template devices (e.g., to generate a DNA template corresponding to the target mRNA sequence), IVT devices (e.g., to generate the target mRNA sequence), formulation devices (e.g., to generate a drug of the target mRNA sequence), or various combinations thereof. The processor chip 104 or other components of the nucleic acid manufacturing system 100 can generate an mRNA therapeutic, such as by encapsulating at least one mRNA with at least one delivery vehicle composition to form the mRNA therapeutic.

The nucleic acid manufacturing system 100 can include at least one controller 108. The controller 108 can be configured to control operation of the processor chip 104. For example, the controller 108 can control various components included in or coupled with the processor chip 104, such as flow control devices (e.g., pumps, valves), heating or cooling elements or fluid flows, optical elements, or other components to control the reactions performed by the processor chip 104. The controller 108 can include one or more processors and memory configured to execute computer-readable instructions (e.g., stored in the memory) to perform various operations described herein, including receiving sensor data from sensors 112 and controlling operation of the processor chip 104 or components thereof using the sensor data.

The nucleic acid manufacturing system 100 can include at least one sensor 112. The at least one sensor 112 can detect one or more parameters of the processor chip 104 or materials in the processor chip 104, such as fluids, reagents, or products 106.

The controller 108 can perform various operations to control generation of the product 106, including based on indications of errors or metrics determined by the metric generator 300 as described further herein. For example, the controller 108 can control operation of the processor chip 104 (or components thereof) using at least one of the indication or a parameter detect by the at least one sensor 112. The controller 108 can control operation of the processor chip 104 to control temperatures, pressures, flow rates of reagents, time of introduction of reagents, duration of reactions or mixing of reagents, or various other process steps performed at one or more paths of the processor chip 104.

The controller 108 can control operation of the processor chip 104 to reduce errors (including, for example, based on identifying contaminants associated with errors) identified from the indication (generated by the metric generator 300). For example, the controller 108 can identify or receive instructions identifying a particular reagent or nucleotide of the product 106 associated with an error, such as based on one or more UMIs associated with the material of the product 106 associated with the error (which can be traced back to the reagent or path to which the UMI(s) were introduced to generate the product 106). The controller 108 can identify or receive instructions identifying particular process steps or paths of the processor chip 104 associated with the error, and modify operation of the processor chip 104 to not use the particular process steps or paths, or reduce flow rates of reagents used in the particular process steps or paths. The controller 108 can identify or receive instructions identifying a particular contaminant of the product 106 associated with the error, and cause the processor chip 104 to provide the product 106 to a purification path to remove the contaminant. For example, the controller 108 can identify or receive instructions identifying a type of the particular contaminant, identify (e.g., from a lookup table or other logical or heuristic data structure maintained in memory of the controller 108) or receive instructions identifying a type of purification to be performed to remove the contaminant, and cause the processor chip 104 to flow the product 106 through the identified purification path to remove the contaminant. The controller 108 can identify or receive instructions identifying a target ratio of nucleic acids of the product 106, determine (from the error or metrics associated with the error) a difference between the target ratio and an actual ratio for the product 106, and modify operation of the processor chip 104 to reduce the difference. The controller 108 can control generation of the product 106 in various control flows and feedback loops using data received from the metric generator 300 or instructions determined using the data outputted by the metric generator 300, such as to iteratively update generation of the product 106 to achieve target metrics or specifications for the product 106.

### B. Nucleic Acid Sequencing

To evaluate characteristics of the product 106, which can be, for example, DNA samples, mRNA samples, or drug products having nucleic acids, the product 106 can undergo sequencing.

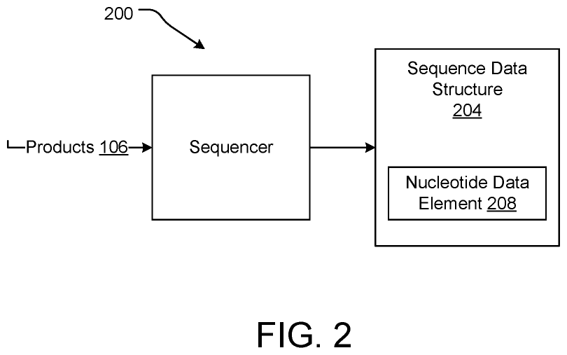

FIG. 2 depicts an example of a sequencer 200. The sequencer 200 can be a next generation sequencing (NGS) system, such as a sequencer that performs parallel sequencing (e.g., by sequencing multiple nucleic acids in parallel). The sequencer 200 can be implemented as a separate device from the nucleic acid manufacturing system 100 and metric generator 300 described herein. The sequencer 200 can receive the product 106 as an input, and output at least one sequence data structure 204 representative of the product 106. The sequence data structure 204 can include a plurality of nucleotide data elements 208, which can represent nucleotides and the order of nucleotides of the nucleic acid(s) of the product 106.

The sequencer 200 can perform various operations to detect the nucleotides of the product 106 to assign to the sequence data structure 204, such as to use a flow cell, amplification, dyeing (e.g., fluorescent dyes), or various combinations thereof. The sequencer 200 can identify each nucleotide of the product 106, and can include one or more processors and memory configured to assign the nucleotide to a respective data element 208 of the sequence data structure 204.

Unique molecular indices (UMIs) (or unique molecular identifiers) can be applied (e.g., ligated) to the product 106 to uniquely tag the starting molecules of a library preparation. The UMI can be a random string of nucleotides that can be ligated onto a molecule of the product 106 prior to other operations of sequencing the product 106, such as prior to PCR being applied to the molecule. The UMIs can be applied to label one or more nucleic acid strands of the molecule, such as to facilitate duplex sequencing using the UMIs and to facilitate tagging each nucleic acid strand using a distinct UMI (which can allow for errors to be detected for specific nucleic acid strands).

### C. Nucleic Acid Sequence Metric Generation

As discussed above, there can be various sources of error in the sequence data structure 204 relative to an actual sequence of the product 106 (e.g., analytical differences, such as computational, PCR, and/or sequencing errors), as well of the product 106 relative to a target sequence intended to be synthesized when manufacturing the product 106 (e.g., biological differences). For example, the product 106 can have misincorporated DNA/RNA bases or contaminating DNA or other possible contaminants; the sequence data structure 204 can be susceptible to factors such as amplification biases or sequencing errors such as a base that is detected by the sequencing system 200 and included in the sequence data structure 204 that does not represent the true or actual base in the underlying product 106, which can, for example, result from a sequencing by synthesis process performed by the sequencing system 200 that becomes out of sync within clonal copies within a cluster, such that some molecules in the cluster can transmit incorrect signals, increasing noise and decreasing accuracy in assigning fluorescence signals to base calls.

Systems and methods in accordance with the present disclosure can address various such computational and biological errors of the sequence data structure 204 and the product 106 by determining one or more metrics of the sequence data structure 204 corresponding to one or more such errors, enabling more accurate generation of the sequence data structure 204. The metrics can be selectively determined in a particular order, reducing computational resources expended to generate the metrics. The metrics can be used to trigger actions such as modifying how the product 106 is synthesized, modifying how the sequence data structure 204 is generated, or various combinations thereof, enabling generation of the product 106 so that the product 106 more closely resembles the target sequence (e.g., does not have errors such as bases that are different than intended in the target sequence). For example, the metrics can be used for various quality control actions.

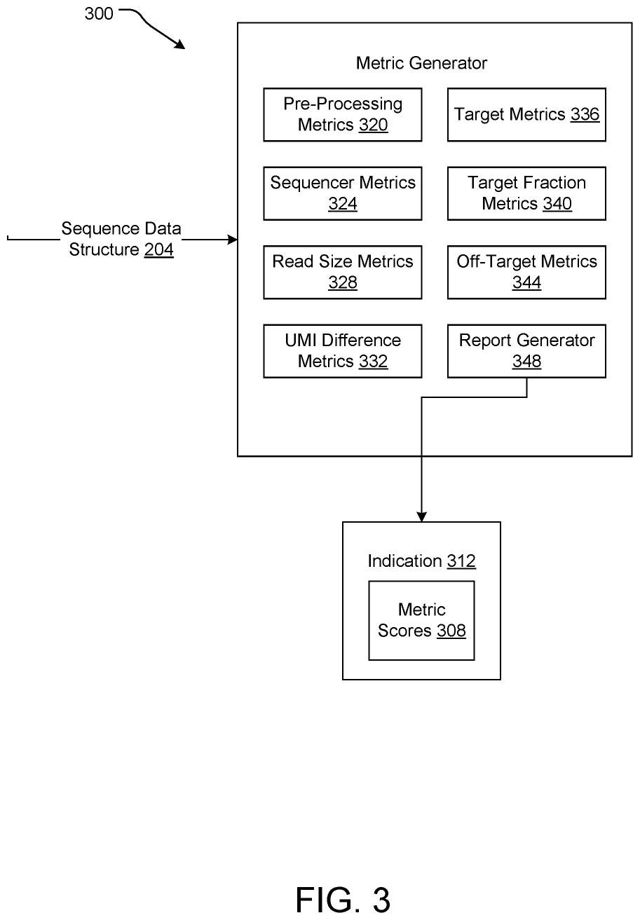

FIG. 3 depicts an example of a metric generator 300. The metric generator 300 can be used to perform various processes as described herein to accurately and efficiently generate metrics regarding nucleic acids, such as to generate metrics based on the sequence data structure 204. The metric generator 300 can be used to evaluate characteristics of a sample of one or more nucleic acids (e.g., of the product 106 from which the sequence data structure 204 is detected) such as a degree to which synthesized nucleic acids in the sample differ from respective target sequences; relative ratios of target sequences in the sample when multiple target sequences are intended; and the prevalence and taxonomic makeup of exogenous nucleic acid contaminants in the sample. The sequence metric generation system 300 can trigger or be implemented with actions to reduce errors represented in the sequence data structure 204, such as to reduce analytical noise, including ligating UMI tags to library molecules prior to amplification, and merging overlapping read pairs to find and resolve sequencing errors. The metric generator 300 can include one or more processors and memory configured to execute computer-readable instructions to perform various operations described herein.

The metric generator 300 can receive the sequence data structure 204. For example, the metric generator 300 can be transmitted the sequence data structure 204, or can access one or more databases in which the sequence data structure 204 is stored or maintained.

The metric generator 300 can apply one or more metrics 304 of a plurality of metrics 304 to the sequence data structure 204 to generate respective metric scores 308 for the metrics 304, and can output an indication 312 of the metric scores 308.

The metrics 304 can be one or more functions, computations, equations, algorithms, filters, rules, heuristics, policies, logic, or other operations implemented by the processor and memory of the metric generator 300, and which can receive the sequence data structure 204 (or a portion of the data of the sequence data structure 204), and generate an output responsive to receiving the sequence data structure 204. As described further herein, the metric generator 300 can selectively apply metrics 304 to the sequence data structure 204, such as in a particular order, and discontinue metric application or otherwise output an error responsive to the applied metrics 304 not satisfying a respective condition (e.g., threshold), which can enable the metric generator 300 to more efficiently (e.g., using fewer computational resources) process the sequence data structure 204 to identify and correct at least one of analytical errors of the sequencing processes used to generate the sequence data structure 204 or biological errors of the product 106.

The metrics 304 can include at least one pre-processing metric 320. The pre-processing metrics 320 can include various filters, metadata extractors, or other metrics that can be used to prepare the data of the sequence data structure 204 for further evaluation. The pre-processing metrics 320 can include a file consistency check metric 320, which can parse the sequence data structure 204 to compare the sequence data structure 204 to an expected file template (e.g., list of expected files, such as XML metadata files). Responsive to the comparison indicating that the sequence data structure 204 does not match the expected file template (e.g., expected files or metadata are missing), the metric generator 300 can output an error.

The metrics 304 can include at least one sequencer metric 324. The sequencer metric 324 can evaluate characteristics of the sequence data structure 204 relating to the sequencing process performed on the product 106 to generate the sequence data structure 204. For example, the sequencer metric 324 can include metrics relating to the flow cell used for sequencing the product, such as whether the flow cell is expired, whether the flow cell was rehybridized, a completion status of the flow cell, whether all planned cycles were completed for all reads, or various combinations thereof. The metric generator 300 can use one or more sequencer metrics 324 as a gating metric to determine whether to evaluate additional metrics. For example, responsive to determining whether one or more sequencer metrics 324 satisfies a target value (e.g., threshold), the metric generator 300 can determine whether to evaluate additional metrics 304.

The sequencer metric 324 can include a flow cell yield metric, which indicates a yield (e.g., total yield in bases of the flow cell) that the metric generator 300 can compare with an expected value or specification for a type of the flow cell (e.g., a value on the order of gigabases (Gb)).

The sequencer metric 324 can include a base quality metric, such as a metric indicating a proportion of bases (e.g., from one or more of reads of the sequence data structure 204) having a quality score that meets or exceeds a threshold. The sequencer metric 324 can indicate a probability of error relative to the threshold. For example, the base quality metric can be a Q30 metric, indicating a fraction of the bases of the sequence data structure 204 having a quality score of at least 30. The quality score can be included in the sequence data structure 204. The metric generator 300 can evaluate a condition regarding the base quality metric by comparing the base quality metric with a threshold value, such as a minimum threshold (e.g., eighty percent).

The metric generator 300 can output an error responsive to the base quality metric being less than the minimum threshold. The metric generator 300 can determine the condition to be satisfied responsive to the base quality metric meeting or exceeding the threshold value, such as to continue with generating various metrics 304 responsive to the base quality metric meeting or exceeding the threshold value.

The sequencer metric 324 can include a filter evaluation metric. For example, the sequence data structure 204 can indicate a number of clusters of the product 106 that passed one or more filters used to generate the sequence data structure 204, such as a filter associated with purity of intensity of the signal detected by the sequencer 200 to identify bases. The metric generator 300 can evaluate a condition regarding the filter evaluation metric by comparing the number of clusters that passed the filters to a threshold value (e.g., a threshold value corresponding to a type of the flow cell, which can be on the order of millions, such as 7 million for a mid-output flow cell or 22 million for a high-output flow cell).

The sequencer metric 324 can include a control library metric. The control library metric can correspond to a control library (e.g., nucleic acid library, such as a non-indexed library, such as the PhiX control library manufactured by Illumina, Inc. of San Diego, CA) that is included with the product 106 during sequencing of the product 106 by the sequencer 200. The sequencer 200 can determine the control library metric by comparing sequence reads detected while generating the sequence data structure 204 with the control library to identify a rate of mismatches (e.g., the control library metric can be an error rate such as the rate of mismatches). The metric generator 300 can evaluate a condition regarding the control library metric by comparing the control library metric to a threshold value (e.g., maximum threshold), and determining the control library metric to satisfy the condition responsive to the control library metric being less than or equal to the threshold value. The metric generator 300 can determine the condition to not be satisfied responsive to the control library metric exceeding the threshold value.

The metrics 304 can include a read size metric 328. The read size metric 328 can correspond to a number of read clusters of the library represented by the sequence data structure 204. The read cluster can represent one or more nucleic acids of the library represented by the sequence data structure 204 that are identified by the sequencer 200 or metric generator 300 as being duplicates. The read size metric 328 can be related to a number of UMI clusters (e.g., read clusters corresponding with unique UMIs) of sufficient depth (e.g., depth greater than or equal to two), as factors such as the molecular complexity (e.g., number of starting DNA molecules) and sequencing depth of the library can affect the number of UMI clusters of sufficient depth, and the number of read clusters can be indicative of such factors.

As such, the metric generator 300 can determine the read size metric 328, compare the read size metric 328 to a threshold value, such as a minimum threshold (e.g., 1 million read clusters), and determine the read size metric 328 to satisfy a condition responsive to the read size metric 328 meeting or exceeding the threshold value. The metric generator 300 can determine the condition to not be satisfied responsive to the read size metric 328 being less than the threshold value, such as to output an error.

As noted above, UMIs can be applied to the molecules of the product 106 prior to sequencing, and thus the UMIs can be indicated in the sequence data structure 204. The metric generator 300 can assign, using the sequence data structure 204, read clusters to corresponding UMI families (each family having a same UMI and position of the UMI in the target sequence). For example, each UMI family can include one or more read clusters having the same UMI at the same position in the target sequence. As such, differences in sequences represented by the sequence data structures 204 can correspond to sources of error such as PCR error or sequencing error, which can be removed to attempt to leave primarily biological differences in the product 106 relative to the target sequence.

For example, the metric generator 300 can apply at least one UMI difference metric 332 to the sequence data structure 204 to identify differences in bases amongst nucleic acids (e.g., UMI clusters) represented by the sequence data structure 204 that differ amongst each other. The metric generator 300 can perform actions such as discarding clusters of the sequence data structure 204 based on the UMI difference metric 332 prior to further evaluation (e.g., to remove PCR errors or sequencing errors), or outputting an error responsive to the UMI difference metric 332 not meeting or exceeding a threshold value (e.g., samples with fewer than a threshold number of UMI clusters that are covered by at least two reads, such as 2000 UMI clusters, result in an error).

The metric generator 300 can determine at least one target metric 336 (e.g., using the output of any of the metrics described herein or combinations thereof as input), such as to determine how closely the sequence data structure 204 matches the target sequence. The metric generator 300 can perform various trimming or merging operations on the sequence data structure 204 prior to determining the at least one target metric 336. For example, the metric generator 300 can identify and remove bases of adapters that were ligated during library preparation. The metric generator 300 can merge R1/R2 read pairs, which can improve the quality of the data of the sequence data structure 204 for generation of the target metrics 336 (e.g., to perform on-target mismatch analysis), and can discard unmerged read pairs. For example, merging overlapping read pairs can lead to lower sequencing error rates by addressing portions of sequences most likely to have sequencing errors (e.g., from the 3′ end of sequences).

The metric generator 300 can align the sequence data structure 204 (e.g., responsive to performing merging) to a reference sequence. The reference sequence can be a predefined sequence representing the sequence that was intended to be synthesized (e.g., target sequence). The metric generator 300 can align the sequence data structure 204 with multiple reference sequences simultaneously (which can improve specificity).

The metric generator 300 can determine the at least one target metric 336 by comparing the sequence data structure 204 (e.g., responsive to aligning the sequence data structure with the reference sequence) with the reference sequence. For example, the metric generator 300 can compare each the base (e.g., A, T, C, G, N, insertion, deletion) of each nucleotide data element 208 of the sequence data structure 204 with the corresponding base (e.g., allele) of the reference sequence, and determine a count of differences based on the comparisons to determine the at least one target metric 336 as a mismatch rate. The metric generator 300 can determine a consensus sequence from a plurality of sequences of sequence data structures 204 by determining a most frequently observed base at each position, and determine the at least one target metric 336 to include a percentage of positions that differ between the consensus sequence and the reference sequence.

The metric generator 300 can determine the at least one target metric 336 to include a mismatch percentage for a particular position (e.g., for each position) as a proportion of alleles that differ at the particular position to a sum of the alleles that differ and the reference allele. The metric generator 300 can determine a target-wide mismatch rate of the at least one target metric 336 to include at least one of a mean mismatch rate (e.g., an average of the mismatch percentages for the particular position), a mean mismatch rate that discounts or does not include mismatch percentages having UMI-covered depths less than a threshold depth (e.g., 200), or a weighted mismatch rate by dividing a number of errors relative to the entire reference sequence by a total number of bases of the reference sequence (e.g., weighting the contribution of error calculation by depth of each position).

The metric generator 300 can determine the at least one target metric 336 to include a target fraction metric 340. The target fraction metric 340 can indicate a relative fraction of the product 106 made up of a particular transcript (e.g., in relation to another transcript, contamination DNA, or various combinations thereof). The metric generator 300 can determine the target fraction metric 340 by counting a number of particular UMIs (e.g., unique, distinct, or discrete UMIs) associated with the particular transcript, and comparing counts of particular UMIs identified from the sequence data structures 204 associated with (e.g., mapping to) the particular transcript with counts of particular UMIs not associated with the particular transcript.

The metric generator 300 can determine at least one off-target metric 344 based on the sequence data structure 204, such as to identify and characterize off-target sequences. Off-target sequences can be non-aligned sequences that do not align with the reference sequences (e.g., with any intended target sequence), which can be due to factors such as contamination by outside cells or nucleic acids, too many mismatches with respect to the reference sequence, or for representing chimeric library preparation artifacts. The metric generator 300 can identify the non-aligned sequences, and compare the non-aligned sequences with one or more off-target reference sequences (e.g., which can be retrieved from various databases or other sequencing runs performed by the sequencer 200).

The metric generator 300 can include a report generator 348. The report generator 348 can generate an output that provides the indication 312 of the metric scores 308, such as to indicate values of the metrics 304 or error conditions resulting from evaluation of the metrics 304. The indication 312 can be used for determining instructions, actions, or other operations for modifying synthesis of nucleic acids, such as modifying synthesis of mRNAs for which the metrics 304 are determined, such as to trigger at least one of re-preparation of the product 106 or re-sequencing of the product 106 (e.g., on a new flow cell).

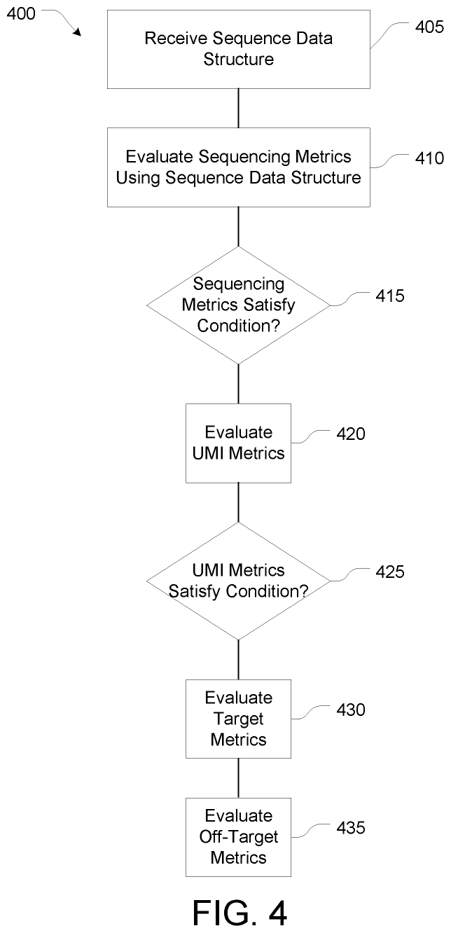

FIG. 4 depicts an example of a method 400 of generating nucleic acid sequencing metrics. The method 400 can be performed using various systems and devices described herein, including but not limited to the sequence metric generation system 300. The method 400 or operations thereof can be performed subsequent or in response to generation of output by a nucleic acid sequencing system (e.g., sequencer 200). The method 400 can be performed during operation of the nucleic acid manufacturing system 100, the sequencer 200, or various combinations thereof, such as to provide outputs of metrics that can be used by control schemes for manufacturing or sequencing nucleic acids in order to reduce analytical, sequencing, or biological errors, such as differences between sequence data representative of a nucleic acid and a target sequence for the nucleic acid. The method 400 can include generation of various metrics described herein (e.g., metrics described with reference to FIG. 3 and the metric generator 300).

At 405, a sequence data structure is received. The sequence data structure can be received responsive to requesting the sequence data structure from a sequencer or a database storing or maintaining the sequence data structure. The sequence data structure can be received in one or more batches or streams of data, such as from various sequencers or databases.

At 410, at least one first metric is evaluated. The first metric can be a metric relating to the sequencing process used to determine the sequence data structure. For example, the first metric can be determined based on data from sequencing components such as a flow cell, such as flow cell expiration, rehybridization, or completion status. The first metric can be a flow cell yield metric. The first metric can be a base quality metric. The first metric can be evaluated to determine whether the data of the sequence data structure is of sufficient quality for further metrics to be determined from the data of the sequence data structure.

At 415, it is determined whether the at least one first metric satisfies a first condition. The first condition can be at least one of a quantitative (e.g., threshold) or qualitative (e.g., category) condition. For example, responsive to a flow cell yield metric score and base quality score each meeting or exceeding a respective threshold, the first condition can be determined to be satisfied. Responsive to the first condition not being satisfied, at least one of an error can be outputted (which can indicate the metric that did not satisfy the first condition) or a modification of a process (e.g., nucleic acid synthesis, nucleic acid sequencing) resulting in the sequence data structure can be performed.

At 420, at least one second metric is evaluated. The at least one second metric can be a metric corresponding to one or more UMIs associated with the sequence data structure, such as UMIs having been ligated to one or more nucleic acids (or strands or portions thereof) represented by the sequence data structure. For example, the at least one second metric can be a UMI difference metric indicating differences in bases amongst UMI portions of nucleic acids represented by the sequence data structure.

At 425, it is determined whether the at least one second metric satisfies a second condition. For example, responsive to the UMI difference metric score being less than a threshold value, it can be determined that there is insufficient data to effectively evaluate further metrics regarding the sequence data structure, and an error can be outputted or other action taken to modify synthesis or sequencing of the nucleic acid.

At 430, at least one target metric can be evaluated. For example, the target metric can be evaluated responsive to the first and second conditions being satisfied (e.g., these conditions can indicate that sequencing data of sufficient volume and quality is available to effectively evaluate target and off-target metrics). The target metric can indicate a match (or mismatch) rate between the sequence data structure and a target sequence. The target metric can be evaluated responsive to at least one of trimming adapters or merging read pairs of the sequence data structure, reducing errors that can be present in the sequence data structure prior to determining the target metrics (which can make the target metrics and any actions triggered based on the target metrics more accurate).

At 435, at least one off-target metric is evaluated. The off-target metric can identify off-target sequences. For example, the off-target metric can be evaluated by identifying sequences that do not align with a reference sequence, comparing the non-aligned sequences with non-reference sequences, and detecting matches between the non-aligned sequences and the non-reference sequences. Counts of non-aligned sequences can be compared with counts of sequences of the sequence data structure to determine the off-target metric (e.g., determine a rate of off-target sequences).

### D. Systems Including Microfluidic Processor Chips

FIG. 5 depicts an example of a system 500, at least some features of which can be used to implement the nucleic manufacturing system 100 described with reference to FIG. 1. The system 500 can include a housing 503 enclosing a seating mount 515 that can removably receive one or more processor chips 511 (e.g., microfluidic process chips). The system 500 can include a chip-receiving component that is configured to removably accommodate the processor chips 511, where the processor chip 511 itself defines one or more microfluidic channels or fluid pathways. Components of the system 500 (e.g., within housing 503) that fluidically interact with the processor chip 511 can include fluid channels or pathways that can not be microfluidic (e.g., with such fluid channels or pathways being larger than the microfluidic channels or fluid pathways in processor chip 511). The processor chips 511 can be provided and utilized as single-use devices, while the rest of system 500 can be reusable. The housing 503 can be in the form of a chamber, enclosure, etc., with an opening that can be closed (e.g., via a lid or door, etc.) to thereby seal the interior. The housing 503 can enclose a thermal regulator and/or can be configured to be enclosed in a thermally-regulated environment (e.g., a refrigeration unit, etc.). The housing 503 can form an aseptic barrier. The housing 503 can form a humidified or humidity-controlled environment. The system 500 can be positioned in a cabinet (not shown). Such a cabinet can provide a temperature-regulated (e.g., refrigerated) environment. Such a cabinet can also provide air filtering and air flow management and can promote reagents being kept at a desired temperature through the manufacturing process. In addition, such a cabinet can be equipped with UV lamps for sterilization of the processor chip 511 and other components of the system 500. Other suitable features can be incorporated into a cabinet that houses the system 500.

The assembly formed by the housing 503 and the components of the system 500 that are within the housing 503, without processor chip 511, can be considered as being an instrument. While the controller 521 and the user interface 523 are depicted in FIG. 5 as being outside of the housing 503, the controller 521 and the user interface 523 can be provided in or on the housing 503 and can thus also form part of the instrument. As described in greater detail below, this instrument can removably receive the processor chip 511 via a seating mount 515. When the processor chip 511 is seated in seating mount 515, the instrument and the processor chip 511 can cooperate to together form the system 500. When the processor chip 511 is removed from the seating mount 515, the portion of the system 500 that is left can be regarded as the instrument. The instrument, the system 500, and the processor chip 511 can each be considered an apparatus.

The seating mount 515 can be configured to secure the processor chip 511 using one or more pins or other components configured to hold the processor chip 511 in a fixed and predefined orientation. The seating mount 515 can thus facilitate the processor chip 511 being held at an appropriate position and orientation in relation to other components of the system 500. The seating mount 515 can hold the processor chip 511 in a horizontal orientation, such that the processor chip 511 is parallel with the ground.

A thermal control 513 can be located adjacent to the seating mount 515, to modulate the temperature of any processor chip 511 mounted in the seating mount 515. The thermal control 513 can include a thermoelectric component (e.g., Peltier device, etc.) and/or one or more heat sinks for controlling the temperature of all or a portion of any processor chip 511 mounted in the seating mount 515. More than one thermal control 513 can be included, such as to separately regulate the temperature of different ones of one or more regions of the processor chip 511. The thermal control 513 can include one or more thermal sensors (e.g., thermocouples, etc.) that can be used for feedback control of the processor chip 511 and/or the thermal control 513.

As depicted in FIG. 5, a fluid interface assembly 509 couples the processor chip 511 with a pressure source 517, thereby providing one or more paths for fluid (e.g., gas) at a positive or negative pressure to be communicated from the pressure source 517 to one or more interior regions of the processor chip 511 as will be described in greater detail below. While only one pressure source 517 is shown, the system 500 can include two or more pressure sources 517. Pressure can be generated by one or more sources other than the pressure source 517. For instance, one or more vials or other fluid sources within reagent storage frame 507 can be pressurized. Reactions and/or other processes carried out on the processor chip 511 can generate additional fluid pressure. The fluid interface assembly 509 can also couple the processor chip 511 with a reagent storage frame 507, thereby providing one or more paths for liquid reagents, etc., to be communicated from reagent storage frame 507 to one or more interior regions of the processor chip 511 as will be described in greater detail below.

Pressurized fluid (e.g., gas) from at least one pressure source 517 can reach fluid interface assembly 509 via reagent storage frame 507, such that reagent storage frame 507 includes one or more components interposed in the fluid path between the pressure source 517 and the fluid interface assembly 509. One or more pressure sources 517 can be directly coupled with the fluid interface assembly 509, such that the positively pressurized fluid (e.g., positively pressurized gas) or negatively pressurized fluid (e.g., suction or other negatively pressurized gas) bypasses the reagent storage frame 507 to reach the fluid interface assembly 509. Regardless of whether the reagent storage frame 507 is interposed in the fluid path between the pressure source 517 and the fluid interface assembly 509, the fluid interface assembly 509 can be removably coupled to the rest of the system 500, such that at least a portion of the fluid interface assembly 509 can be removed for sterilization between uses. As described in greater detail below, the pressure source 517 can selectively pressurize one or more chamber regions on the processor chip 511. The pressure source 517 can also selectively pressurize one or more vials or other fluid storage containers held by the reagent storage frame 507.

The reagent storage frame 507 can contain a plurality of fluid sample holders, each of which can hold a fluid vial that is configured to hold a reagent (e.g., nucleotides, solvent, water, etc.) for delivery to the processor chip 511. One or more fluid vials or other storage containers in the reagent storage frame 507 can receive a product from the interior of the processor chip 511. A second processor chip 511 can receive a product from the interior of a first processor chip 511, such that one or more fluids are transferred from one processor chip 511 to another processor chip 511. The first processor chip 511 can perform a first dedicated function (e.g., synthesis, etc.) while the second processor chip 511 performs a second dedicated function (e.g., encapsulation, etc.). The reagent storage frame 507 can include a plurality of pressure lines and/or a manifold configured to divide one or more pressure sources 517 into a plurality of pressure lines that can be applied to the processor chip 511. Such pressure lines can be independently or collectively (in sub-combinations) controlled.

The fluid interface assembly 509 can include a plurality of fluid lines and/or pressure lines where each such line includes a biased (e.g., spring-loaded) holder or tip that individually and independently drives each fluid and/or pressure line to the processor chip 511 when the processor chip 511 is held in the seating mount 515. Any associated tubing (e.g., the fluid lines and/or the pressure lines) can be part of the fluid interface assembly 509 and/or can connect to the fluid interface assembly 509. Each fluid line can include a flexible tubing that connects between reagent storage frame 507, via a connector that couples the vial to the tubing in a locking engagement (e.g., ferrule) and the processor chip 511. The ends of the fluid lines/pressure lines can be configured to seal against the processor chip 511 (e.g., at a corresponding sealing port formed in the processor chip 511), as described below. The connections between the pressure source 517 and the processor chip 511, and the connections between vials in the reagent storage frame 507 and the processor chip 511, all form sealed and closed paths that are isolated when the processor chip 511 is seated in the seating mount 515. Such sealed, closed paths can provide protection against contamination when processing therapeutic polynucleotides.

The vials of the reagent storage frame 507 can be pressurized (e.g., >1 atm pressure, such as 2 atm, 3 atm, 5 atm, or higher). In some versions, the vials can be pressurized by the pressure source 517. Negative or positive pressure can thus be applied. For example, the fluid vials can be pressurized to between about 1 and about 20 psig (e.g., 5 psig, 10 psig, etc.). A vacuum (e.g., about −7 psig or about 7 psia) can be applied to draw fluids back into the vials (e.g., vials serving as storage depots) at the end of the process. The fluid vials can be driven at lower pressure than the pneumatic valves as described below, which can prevent or reduce leakage. The difference in pressure between the fluid and pneumatic valves can be between about 1 psi and about 25 psi (e.g., about 3 psi, about 5 psi, 7 psi, 10 psi, 12 psi, 15 psi, 20 psi, etc.).

System 500 can include a magnetic field applicator 519, which is configured to create a magnetic field at a region of the processor chip 511. The magnetic field applicator 519 can include a movable head that is operable to move the magnetic field to thereby selectively isolate products that are adhered to magnetic capture beads within vials or other storage containers in the reagent storage frame 507.

The system 500 can include one or more sensors 505. The sensors 505 can include one or more cameras and/or other kinds of optical sensors. The sensors 505 can sense one or more of a barcode, a fluid level within a fluid vial held within the reagent storage frame 507, fluidic movement within a processor chip 511 that is mounted within the seating mount 515, and/or other optically detectable conditions. The sensor 505 can be used to sense barcodes included on vials of the reagent storage frame 507, such that the sensor 505 can be used to identify vials in the reagent storage frame 507. A single sensor 505 can be positioned and configured to simultaneously view such barcodes on vials in the reagent storage frame 507, fluid levels in vials in the reagent storage frame 507, fluidic movement within a processor chip 511 that is mounted within the seating mount 515, and/or other optically detectable conditions. More than one sensor 505 is used to view such conditions. Different sensors 505 can be positioned and configured to separately view corresponding optically detectable conditions, such that a sensor 505 can be dedicated to a particular corresponding optically detectable condition.

In versions where sensors 505 include at least one optical sensor, visual/optical markers can be used to estimate yield. For example, fluorescence can be used to detect process yield or residual material by tagging with fluorophores. Dynamic light scattering (DLS) can be used to measure particle size distributions within a portion of the processor chip 511 (e.g., such as a mixing portion of the processor chip 511). The sensor 505 can provide measurements using one or two optical fibers to convey light (e.g., laser light) into the processor chip 511 and detect an optical signal coming out of the processor chip 511. To detect, for example, process yield or residual material, etc., the sensor 505 can be configured to detect visible light, fluorescent light, an ultraviolet (UV) absorbance signal, an infrared (IR) absorbance signal, and/or any other suitable kind of optical feedback.

In examples where sensors 505 include at least one optical sensor that is configured to capture video images, the sensors 505 can record at least some activity on the processor chip 511. For example, an entire run for synthesizing and/or processing a material (e.g., a therapeutic RNA) can be recorded by one or more video sensors 505, including a video sensor 505 that can visualize the processor chip 511 (e.g., from above). Processing on the processor chip 511 can be visually tracked and this video record can be retained for later quality control and/or processing. Thus, the video record of the processing can be saved, stored, and/or transmitted for subsequent review and/or analysis. In addition, as will be described in greater detail below, the video can be used as a real-time feedback input that can affect processing using at least visually observable conditions captured in the video.

The system 500 of the present example can be controlled by a the controller 521. The controller 521 can include one or more processors, one or more memories, and various other suitable electrical components. One or more components of the controller 521 (e.g., one or more processors, etc.) can be embedded within the system 500 (e.g., contained within the housing 503). One or more components of the controller 521 (e.g., one or more processors, etc.) can be detachably attached or detachably connected with other components of the system 500. Thus, at least a portion of the controller 521 can be removable. Moreover, at least a portion of the controller 521 can be remote from the housing 503.

The control by the controller 521 can include activating the pressure source 517 to apply pressure through processor chip 511 to drive fluidic movement. The controller 521 can be completely or partially outside of the housing 503; or completely or partially inside of the housing 503. The controller 521 can be configured to receive user inputs via a the user interface 523 of the system 500, and provide outputs to users via the user interface 523. The controller 521 can be fully automated to a point where user inputs are not needed, such that the user interface 523 provides only outputs to users. The user interface 523 can include a monitor, a touchscreen, a keyboard, and/or any other suitable features. The controller 521 can coordinate processing, including moving one or more fluid(s) onto and on the processor chip 511, mixing one or more fluids on the processor chip 511, adding one or more components to the processor chip 511, metering fluid in the processor chip 511, regulating the temperature of the processor chip 511, applying a magnetic field (e.g., when using magnetic beads), etc. The controller 521 can receive real-time feedback from sensors 505 and execute control algorithms in accordance with such feedback from sensors 505. Such feedback from sensors 505 can include, but need not be limited to, identification of reagents in vials in the reagent storage frame 507, detected fluid levels in vials in the reagent storage frame 507, detected movement of fluid in the processor chip 511, fluorescence of fluorophores in fluid in the processor chip 511, etc. The controller 521 can include software, firmware and/or hardware. The controller 521 can also communicate with a remote server, e.g., to track operation of the apparatus, to re-order materials (e.g., components such as nucleotides, processor chips 511, etc.), and/or to download protocols, etc.

It should be appreciated that all combinations of the foregoing concepts and additional concepts discussed in greater detail below are contemplated as being part of the inventive subject matter disclosed herein and may be employed in any combination to achieve the benefits described herein.

All or part of the processes described herein and their various modifications (hereinafter referred to as “the processes”) can be implemented, at least in part, via a computer program product, i.e., a computer program tangibly embodied in one or more tangible, physical hardware storage devices that are computer and/or machine-readable storage devices for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Processors suitable for the execution of a computer program include, by way of example, both general and special purpose microprocessors, and any one or more processors of any kind of digital computer. Generally, a processor will receive instructions and data from a read-only storage area or a random access storage area or both. Elements of a computer (including a server) include one or more processors for executing instructions and one or more storage area devices for storing instructions and data. Generally, a computer will also include, or be operatively coupled to receive data from, or transfer data to, or both, one or more machine-readable storage media, such as mass storage devices for storing data, e.g., magnetic, magneto-optical disks, or optical disks.

Computer program products are stored in a tangible form on non-transitory computer readable media and non-transitory physical hardware storage devices that are suitable for embodying computer program instructions and data. These include all forms of non-volatile storage, including by way of example, semiconductor storage area devices, e.g., EPROM, EEPROM, and flash storage area devices; magnetic disks, e.g., internal hard disks or removable disks; magneto-optical disks; and CD-ROM and DVD-ROM disks and volatile computer memory, e.g., RAM such as static and dynamic RAM, as well as erasable memory, e.g., flash memory and other non-transitory devices.

The construction and arrangement of the systems and methods as shown in the various embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced. Other substitutions, modifications, changes, and omissions can be made in the design, operating conditions and arrangement of embodiments without departing from the scope of the present disclosure.

As utilized herein, the terms “approximately,” “about,” “substantially”, and similar terms are intended to include any given ranges or numbers +/−10%. These terms include insubstantial or inconsequential modifications or alterations of the subject matter described and claimed are considered to be within the scope of the disclosure as recited in the appended claims.

The term “coupled” and variations thereof, as used herein, means the joining of two members directly or indirectly to one another. Such joining can be stationary (e.g., permanent or fixed) or moveable (e.g., removable or releasable). Such joining can be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling can be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element can be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements can differ according to other exemplary embodiments, and that such variations are intended to be encompassed by the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps can differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

1. A method of generating nucleic acid sequencing metrics, comprising:
receiving, by one or more processors, a sequence data structure, wherein the sequence data structure is from parallel processing of a nucleic acid;
applying, by the one or more processors, at least one first metric of a plurality of metrics to the sequence data structure to generate at least one first metric score;
determining, by the one or more processors, that the at least one first metric score satisfies a first condition;
applying, by the one or more processors, responsive to determining that the at least one first metric scores satisfies the first condition, at least one second metric of the plurality of metrics to the sequence data structure to generate at least one second metric score; and
outputting, by the one or more processors, an indication of the at least one first metric score and the at least one second metric score.

2. The method of claim 1, comprising:
detecting, by the one or more processors, at least one unique molecular index of the sequence data structure; and
generating, by the one or more processors, the at least one second metric score using the at least one unique molecular index.

3. The method of claim 2, wherein the nucleic acid comprises mRNA or DNA, and generating the at least one second metric score comprises adjusting the at least one second metric score to reduce an error rate associated with the sequence data structure to perform quality control of the nucleic acid.

4. The method of claim 2, wherein the at least one unique molecular index is adjacent to an i7 index of the sequence data structure.

5. The method of claim 1, comprising:
identifying, by the one or more processors, at least one synthetic adapter sequence of the sequence data structure;
removing, by the one or more processors, the at least one synthetic adapter sequence from the sequence data structure;
merging, by the one or more processors responsive to removing the at least one synthetic adapter sequence, a plurality of unique molecular indices of the sequence data structure from which the at least one synthetic adapter sequence is removed; and
generating, by the one or more processors, responsive to merging the plurality of unique molecular indices, the at least one second metric score to include a mismatch rate.

6. The method of claim 1, wherein the at least one first metric comprises at least one metric of a flow cell used to generate the sequence data structure.

7. The method of claim 1, comprising outputting, by the one or more processors, an indication of a failure condition responsive to the at least one first metric score not satisfying the first condition.

8. The method of claim 7, wherein outputting the indication comprises generating, by the one or more processors, the indication to include instructions for modifying synthesis of a target sequence from which the sequence data structure is detected.

9. (canceled)

10. The method of claim 1, wherein the at least one second metric score comprises a target mismatch rate corresponding to an amount of mismatch between the sequence data structure and a data structure of a target sequence for synthesis of the nucleic acid.

11. (canceled)

12. The method of claim 1, further comprising evaluating one or more characteristics of an mRNA therapeutic that corresponds to the nucleic acid using the indication.

13-16. (canceled)

17. A system, comprising:
one or more processors to:
receive a sequence data structure, wherein the sequence data structure is from parallel processing of a nucleic acid;
apply at least one first metric of a plurality of metrics to the sequence data structure to generate at least one first metric score;
determine that the at least one first metric score satisfies a first condition;
apply, responsive to the at least one first metric score satisfying the first condition, at least one second metric of the plurality of metrics to the sequence data structure to generate at least one second metric score; and
output an indication of the at least one first metric score and the at least one second metric score.

18. The system of claim 17, wherein the one or more processors are to:
detect at least one unique molecular index of the sequence data structure; and
generate the at least one second metric score using the at least one unique molecular index.

19. (canceled)

20. (canceled)

21. The system of claim 17, wherein the one or more processors are to:
identify at least one synthetic adapter sequence of the sequence data structure;
remove the at least one synthetic adapter sequence from the sequence data structure;
merge, responsive to removing the at least one synthetic adapter sequence, a plurality of unique molecular indices of the sequence data structure from which the at least one synthetic adapter sequence is removed; and
generate, responsive to merging the plurality of unique molecular indices, the at least one second metric score to include a mismatch rate of the sequence data structure relative to a target data structure for synthesis of the nucleic acid.

22-27. (canceled)

28. The system of claim 17, wherein the one or more processors are to control operation of a processor chip to generate an mRNA therapeutic using the indication.

29. The system of claim 28, wherein the one or more processors are to control operation of the processor chip to reduce a difference between the mRNA and a target sequence of the mRNA using the indication.

30. The system of claim 28, wherein the one or more processors are to execute a process to encapsulate at least one mRNA with at least one delivery vehicle composition to form the mRNA therapeutic.

31. The system of claim 17, wherein the one or more processors are to generate a report containing the indication.

32-56. (canceled)

57. The method of claim 1, wherein the nucleic acid is a first nucleic acid, the sequence data structure is from parallel processing of the first nucleic acid and a second nucleic acid, and the method comprises determining, by the one or more processors, in the sequence data structure, a ratio of the first nucleic acid and the second nucleic acid.

58. The method of claim 1, wherein the at least one first metric comprises at least one of a flow cell metric or a base quality metric, and the at least one second metric comprises a target metric of a match rate between the sequence data structure and a target sequence of the nucleic acid.

59. The method of claim 1, comprising:
aligning, by the one or more processors, the sequence data structure with a target sequence; and
determining, by the one or more processors responsive to aligning the sequence data structure with the target sequence, the indication based on comparing the sequence data structure with the target sequence.
